SEQUENCE LISTING (13 sequences)

SEQ ID NO: 1 - protein, 73 aa, Artificial Sequence
  Description of Artificial Sequence Synthetic polypeptide
MGKKPKSSFNVNQDDIPEVIRDNPAGSPQSQPLSPIPLKLIAAGLGIIILALLTLLALWPRPEPAPEVVTEPS

SEQ ID NO: 2 - protein, 69 aa, Artificial Sequence
  Description of Artificial Sequence Synthetic polypeptide
MTEPPVLHETSSESEKEQSIGKQNLSFQPIPQASKPGKRLWLVVGALLLLGGGGYWWFQSRSGGPPGGA

SEQ ID NO: 3 - protein, 97 aa, Artificial Sequence
  Description of Artificial Sequence Synthetic polypeptide
MQAPTQSGGLSLRNKAVLIALLIGLIPAGVIGGLNLSSVDRLPVPQTEQQVKDSTTKQIRDQILIGLLVTAVGAAFVAYWMVGENTKAQTALALKAK

SEQ ID NO: 4 - protein, 116 aa, Artificial Sequence
  Description of Artificial Sequence Synthetic polypeptide
MFLGWFTNASLFRKQIYMAIASGVFSGFAVLVLGSIVGLGGTPKDVPAPSGETTTEAPAEGAPAEGQAPSQTPEEEPGKPSLLNLAFLTAIATAIGVFLINRLLMQQIKSIIDDLQ

SEQ ID NO: 5 - DNA, 219 bp, Artificial Sequence
  Description of Artificial Sequence Synthetic polynucleotide
atgggtaaaaaaccaaaatcttcctttaacgtcaaccaggatgacatcccggaagtaattcgggacaatccagcggggtcgccccagtcccaacccttgtcccccataccgctgaaattaatcgcggccggcttggggataattattctggctttattgacgttgttggccctgtggcccaggcccgagcccgcaccggaagtggtgacagaaccaagt

SEQ ID NO: 6 - DNA, 207 bp, Artificial Sequence
  Description of Artificial Sequence Synthetic polynucleotide
atgactgagccacccgtactgcacgaaacttcttcggaatcggaaaaggaacaaagtattggtaaacagaatttgtcctttcaacccattccccaagcctccaaaccaggcaaacgactctggctagtggtgggagccttactgttgctaggaggtggtggctactggtggtttcagtcccgttccggcggccctcccggaggggcc

SEQ ID NO: 7 - DNA, 291 bp, Artificial Sequence
  Description of Artificial Sequence Synthetic polynucleotide
atgcaggcacctacccaaagtggaggactttccctccgcaataaagctgtgttgatagccctgttaattggtttgatccccgctggggtgattggagggctcaatctcagcagtgtggacaggcttccagtgccccaaacggaacaacaggtcaaggactccaccactaagcaaatccgtgaccaaattctgattgggcttttggtgaccgcggtgggggctgccttcgttgcctactggatggtgggggaaaataccaaagctcaaaccgccctggccttgaaagctaag

SEQ ID NO: 8 - DNA, 348 bp, Artificial Sequence
  Description of Artificial Sequence Synthetic polynucleotide
atgtttctcggttggttcaccaatgcttccctgtttagaaagcagatttacatggcgatcgcctccggtgtgttttccgggttcgcggtgttggtattgggaagcatagtgggtttagggggaactcccaaagacgtacctgctccttcaggggaaaccaccaccgaagctccagcagaaggtgcccccgcagagggccaggccccttcccagaccccagaggaagaaccgggcaaaccatccctcctcaacctcgccttcctcacagccatagccacggcgatcggagtctttctgatcaatcgattgctgatgcaacagattaagagtattatcgacgacctgcaa

SEQ ID NO: 9 - protein, 464 aa, Artificial Sequence
  Description of Artificial Sequence Synthetic polypeptide
MQAPTQSGGLSLRNKAVLIALLIGLIPAGVIGGLNLSSVDRLPVPQTEQQVKDSTTKQIRDQILIGLLVTAVGAAFVAYWMVGENTKAQTALALKAKFHRLWTLIRKELQSLLREPQTRAILILPVLIQVILFPFAATLEVTNATIAIYDEDNGEHSVELTQRFARASAFTHVLLLKSPQEIRPTIDTQKALLLVRFPADFSRKLDTFQTAPLQLILDGRNSNSAQIAANYLQQIVKNYQQELLEGKPKPNNSELVVRNWYNPNLDYKWFVVPSLIAMITTIGVMIVTSLSVAREREQGTLDQLLVSPLTTWQIFIGKAVPALIVATFQATIVLAIGIWAYQIPFAGSLALFYFTMVIYGLSLVGFGLLISSLCSTQQQAFIGVFVFMMPAILLSGYVSPVENMPVWLQNLTWINPIRHFTDITKQIYLKDASLDIVWNSLWPLLVITATTGSAAYAMFRRKVM

SEQ ID NO: 10 - protein, 483 aa, Artificial Sequence
  Description of Artificial Sequence Synthetic polypeptide
MFLGWFTNASLFRKQIYMAIASGVFSGFAVLVLGSIVGLGGTPKDVPAPSGETTTEAPAEGAPAEGQAPSQTPEEEPGKPSLLNLAFLTAIATAIGVFLINRLLMQQIKSIIDDLQFHRLWTLIRKELQSLLREPQTRAILILPVLIQVILFPFAATLEVTNATIAIYDEDNGEHSVELTQRFARASAFTHVLLLKSPQEIRPTIDTQKALLLVRFPADFSRKLDTFQTAPLQLILDGRNSNSAQIAANYLQQIVKNYQQELLEGKPKPNNSELVVRNWYNPNLDYKWFVVPSLIAMITTIGVMIVTSLSVAREREQGTLDQLLVSPLTTWQIFIGKAVPALIVATFQATIVLAIGIWAYQIPFAGSLALFYFTMVIYGLSLVGFGLLISSLCSTQQQAFIGVFVFMMPAILLSGYVSPVENMPVWLQNLTWINPIRHFTDITKQIYLKDASLDIVWNSLWPLLVITATTGSAAYAMFRRKVM

SEQ ID NO: 11 - protein, 473 aa, Artificial Sequence
  Description of Artificial Sequence Synthetic polypeptide
MQAPTQSGGLSLRNKAVLIALLIGLIPAGVIGGLNLSSVDRLPVPQTEQQVKDSTTKQIRDQILIGLLVTAVGAAFVAYWMVGENTKAQTALALKAKSNPILSWRRVRALCVKETRQIVRDPSSWLIAVVIPLLLLFIFGYGINLDSSKLRVGILLEQRSEAALDFTHTMTGSPYIDATISDNRQELIAKMQAGKIRGLVVIPVDFAEQMERANATAPIQVITDGSEPNTANFVQGYVEGIWQIWQMQRAEDNGQTFEPLIDVQTRYWFNPAAISQHFIIPGAVTIIMTVIGAILTSLVVAREWERGTMEALLSTEITRTELLLCKLIPYYFLGMLAMLLCMLVSVFILGVPYRGSLLILFFISSLFLLSTLGMGLLISTITRNQFNAAQVALNAAFLPSIMLSGFIFQIDSMPAVIRAVTYIIPARYFVSTLQSLFLAGNIPVVLVVNVLFLIASAVMFIGLTWLKTKRRLD

SEQ ID NO: 12 - protein, 492 aa, Artificial Sequence
  Description of Artificial Sequence Synthetic polypeptide
MFLGWFTNASLFRKQIYMAIASGVFSGFAVLVLGSIVGLGGTPKDVPAPSGETTTEAPAEGAPAEGQAPSQTPEEEPGKPSLLNLAFLTAIATAIGVFLINRLLMQQIKSIIDDLQSNPILSWRRVRALCVKETRQIVRDPSSWLIAVVIPLLLLFIFGYGINLDSSKLRVGILLEQRSEAALDFTHTMTGSPYIDATISDNRQELIAKMQAGKIRGLVVIPVDFAEQMERANATAPIQVITDGSEPNTANFVQGYVEGIWQIWQMQRAEDNGQTFEPLIDVQTRYWFNPAAISQHFIIPGAVTIIMTVIGAILTSLVVAREWERGTMEALLSTEITRTELLLCKLIPYYFLGMLAMLLCMLVSVFILGVPYRGSLLILFFISSLFLLSTLGMGLLISTITRNQFNAAQVALNAAFLPSIMLSGFIFQIDSMPAVIRAVTYIIPARYFVSTLQSLFLAGNIPVVLVVNVLFLIASAVMFIGLTWLKTKRRLD

SEQ ID NO: 13 - protein, 6 aa, Artificial Sequence
  Description of Artificial Sequence Synthetic 6xHis tag
HHHHHH

(19) United States
(12) Patent Application Publication
(10) Pub. No.: US 2013/0280780 A1
(43) Pub. Date: Oct. 24, 2013

(54) Methods and Compositions for Targeting Heterologous Integral Membrane Proteins to the Cyanobacterial Plasma Membrane

(71) Applicant: JOULE UNLIMITED TECHNOLOGIES, INC., Bedford, MA (US)

(72) Inventor: Nikos B. Reppas, Cambridge, MA (US)

(21) Appl. No.: 13/920,662

(22) Filed: Jun. 18, 2013

Related U.S. Application Data
(63) Continuation of application No. 13/232,945, filed on Sep. 14, 2011.
(60) Provisional application No. 61/382,917, filed on Sep. 14, 2010.
(60) Provisional application No. 61/414,877, filed on Nov. 17, 2010.
(60) Provisional application No. 61/416,713, filed on Nov. 23, 2010.
(60) Provisional application No. 61/478,045, filed on Apr. 21, 2011.

(51) Int. Cl. C12N 15/74; C12P 5/02

(52) U.S. Cl. CPC C12N 15/74; C12P 5/02
USPC 435/167; 435/320.1; 435/257.2; 435/471; 530/350; 536/23.4

(57) ABSTRACT

This disclosure pertains to the functional localization of heterologous integral plasma membrane proteins (HIPMPs) lacking cleavable signal sequences into the plasma membrane (PM) of cyanobacterial hosts, e.g., JCC138 (Synechococcus sp. PCC 7002) or an engineered derivative thereof. More specifically, the disclosure provides chimeric integral plasma membrane proteins comprising pseudo leader sequences (PLSs) that promote increased hydrocarbon (e.g., alkane) export capabilities when expressed in a photosynthetic organism, e.g., a cyanobacterium.

### CROSS-REFERENCE TO RELATED APPLICATIONS

This application claims priority to U.S. Ser. No. 13/232,945, filed Sep. 14, 2011; U.S. Provisional Patent Application No. 61/382,917, filed Sep. 14, 2010; U.S. Provisional Patent Application No. 61/414,877, filed Nov. 17, 2010; U.S. Provisional Patent Application No. 61/416,713, filed Nov. 23, 2010; and U.S. Provisional Patent Application No. 61/478,045, filed Apr. 21, 2011; each of which is herein incorporated by reference in its entirety for all purposes.

This application in addition incorporates by reference the disclosures of U.S. Provisional Patent Application No. 61/224,463 filed, Jul. 9, 2009; U.S. Provisional Patent Application No. 61/228,937, filed Jul. 27, 2009; U.S. utility application Ser. No. 12/759,657, filed Apr. 13, 2010 (now U.S. Pat. No. 7,794,969); and U.S. utility application Ser. No. 12/833,821, filed Jul. 9, 2010.

### SEQUENCE LISTING

The instant application contains a Sequence Listing which has been submitted in ASCII format via EFS-Web and is hereby incorporated by reference in its entirety. Said ASCII copy, created on Dec. 1, 2011, is named 19469US_CRF_sequencelisting.txt and is 23 kb in size.

### BACKGROUND

Previously, recombinant photosynthetic microorganisms have been engineered to produce hydrocarbons, including alkanes, in amounts that exceed the levels produced naturally by the organism. A need exists for engineered photosynthetic microorganisms which have capabilities such that greater amounts of the biosynthetic hydrocarbon products are secreted into the culture medium, thereby minimizing downstream processing steps.

### SUMMARY

This disclosure pertains, in part, to the functional localization of heterologous integral plasma membrane proteins (HIPMPs) lacking cleavable signal sequences to the plasma membrane (PM) of cyanobacterial hosts, e.g., JCC138 (Synechococcus sp. PCC 7002) or an engineered derivative thereof. More specifically, the disclosure provides chimeric integral plasma membrane proteins comprising pseudo leader sequences (PLSs) that promote increased hydrocarbon (e.g., alkane) export capability when expressed in a photosynthetic organism, e.g., a cyanobacterium.

This disclosure also pertains to the recombinant expression of a multi-subunit prokaryotic efflux pump native to E. coli that is capable of mediating the export of intracellular n-alkanes, e.g., n-pentadecane and n-heptadecane, generated by the concerted action of acyl-ACP reductase (Aar) and alkanal deformylative monooxygenase (Adm), and to the heterologous expression of its corresponding structural genes in a photosynthetic microorganism, e.g., a JCC138-derived adm-aar⁺ alkanogen, so as to enable said photosynthetic microorganism host to efflux n-alkanes into the growth medium. Alkenes may also be created and secreted by microbes comprising these enzymes.

The present disclosure also provides, in certain embodiments, isolated or recombinant polynucleotides comprising or consisting of nucleic acid sequences selected from the group consisting of coding sequences for the proteins whose SEQ ID NOs are provided as SEQ ID NOs 9-12, including nucleic acid sequences that are codon-optimized for expressing these proteins in a cyanobacterium. In certain embodiments, the disclosure also provides isolated or recombinant polynucleotides comprising or consisting of nucleic acid sequences selected from the group consisting of coding sequences for the proteins with at least 50%, at least 60%, at least 70%, at least 80%, at least 90%, at least 95%, at least 96%, at least 97%, at least 98%, or at least 99% identity to the proteins whose SEQ ID NOs are provided as SEQ ID NOs 9-12, where the encoded proteins are capable of mediating the export of intracellular n-alkanes when recombinantly expressed in a cyanobacterium.

In another embodiment, the disclosure provides a method for modifying a HIPMP to improve its functionality in a target cyanobacterial cell, wherein said method comprises: (i) fusing a pseudo leader sequence (PLS) to the N-terminus of said HIPMP, wherein said HIPMP has, in its native state, its N-terminus within the cytoplasm, and wherein the PLS consists of two transmembrane alpha helices and a single periplasmic loop sequence linking the two transmembrane alpha helices; or (ii) adding a PLS to the N-terminus of said HIPMP, wherein said HIPMP has, in its native state, its N-terminus within the periplasm, and wherein the PLS consists of a single transmembrane alpha helix. In a related embodiment, where the PLS consists of two transmembrane alpha helices and a single periplasmic loop sequence linking the two transmembrane alpha helices, the PLS is at least 90% identical to a pair of transmembrane alpha helices of an integral plasma membrane protein (IPMP) native to a non-target cyanobacterial species, wherein said IPMP and said pair of transmembrane alpha helices each has, in its native state, its N-terminus within the cytoplasm and its C-terminus within the cytoplasm. In another related embodiment, wherein the PLS consists of a single transmembrane alpha helix, said single transmembrane alpha helix is at least 90% identical to a transmembrane alpha helix of an IPMP native to a non-target cyanobacterial species, wherein said IPMP and said transmembrane alpha helix of said IPMP each has, in its native state, its N-terminus within the cytoplasm and its C-terminus within the periplasm.

In a related embodiment, the target cyanobacterial cell is a Synechococcus species. In another related embodiment, the Synechococcus species is Synechococcus sp. PCC 7002. In another related embodiment of the method, the non-target cyanobacterial cell from which PLS is derived is a Synechocystis species. In yet another related embodiment of the method, the Synechocystis species is Synechocystis sp. PCC 6803. In a related embodiment, the target cyanobacterial cell is a thermophile. In another related embodiment, the non-target cyanobacterial cell from which the PLS is derived is a thermophile.

In yet another related embodiment, the target cyanobacterial cell is a species of Synechococcus engineered to produce increased amounts of hydrocarbons relative to the native species.

In another related embodiment, the HIPMP comprises a cytoplasmic N-terminus in its native state. In another related embodiment, the PLS is selected from the group consisting of SEQ ID NO:1 and SEQ ID NO:2. In another related embodiment of the method, the PLS is at least 80%, 85%, 90%, or 95% identical to SEQ ID NOs: 1 or 2.

In another related embodiment of the method, the HIPMP comprises a periplasmic N-terminus in its native state. In yet another related embodiment, the PLS is selected from the group consisting of SEQ ID NO:3 and SEQ ID NO:4. In another related embodiment of the method, the PLS is at least 80%, at least 85%, at least 90%, or at least 95% identical to SEQ ID NOs: 3 or 4. In another related embodiment, the HIPMP is selected from the group consisting of SEQ ID NOs: 9, 10, 11, and 12. In other related embodiment, the HIPMP is at least 80%, at least 85%, at least 90%, or at least 95% identical to SEQ ID NOs: 9, 10, 11, or 12.

In another embodiment, the present disclosure provides a chimeric integral plasma membrane protein (CIPMP) for facilitating hydrocarbon efflux in a target bacterium, wherein said CIPMP comprises, at its N-terminus, a pseudo leader sequence, wherein said pseudo leader sequence is covalently fused to a heterologous IPMP, and wherein said pseudo leader sequence comprises at least one but no more than two transmembrane alpha helices, and wherein the N-terminus of said CIPMP is in the cytoplasm when expressed in said target bacterium. In a related embodiment, said pseudo leader sequence is identical or homologous to one or two transmembrane alpha helices from a non-target bacterial IPMP. In yet another related embodiment, said IPMP is identical or homologous to a non-target IPMP. In yet another related embodiment, said IPMP is at least 90% identical to a non-cyanobacterial IPMP and said pseudo leader sequence is at least 90% identical to a non-target cyanobacterial integral IPMP. In yet another related embodiment, wherein the IPMP, in its native state, has its N-terminus in the cytoplasm, the pseudo leader sequence comprises two transmembrane alpha helices and a periplasmic loop. In yet another related embodiment, wherein the IPMP, it its native state, has its N-terminus in the periplasm, the pseudo leader sequence comprises a single transmembrane alpha helix.

In yet another embodiment, the pseudo leader sequence comprises two transmembrane helices and a periplasmic loop and is at least 90% identical, at least 95% identical, at least 99% identical, or 100% identical to SEQ ID NO: 3 or 4. In yet another embodiment, the pseudo leader sequence comprises a single transmembrane helix and is at least 90% identical, at least 95% identical, at least 99% identical, or 100% identical to SEQ ID NO: 1 or 2. In another related embodiment, the disclosure provides a chimeric protein selected from the group consisting of SEQ ID NOs: 9, 10, 11, and 12. In other related embodiment, the HIPMP is at least 80%, at least 85%, at least 90%, or at least 95% identical to SEQ ID NOs 9, 10, 11, or 12.

In a related embodiment of the chimeric protein, the non-cyanobacterial integral plasma membrane protein is native to E. coli. In yet another related embodiment, the non-cyanobacterial integral plasma membrane protein is selected from the group consisting of YbhR and YbhS from E. coli MG1655.

In another embodiment, the disclosure provides a recombinant nucleic acid encoding any of the chimeric proteins described in the preceding paragraphs in the Summary. In yet another embodiment, the disclosure provides a recombinant nucleic acid encoding a chimeric protein comprising SEQ ID NO: 1, SEQ ID NO:2, SEQ ID NO:3, or SEQ ID NO:4. In yet another related embodiment, the disclosure provides a recombinant nucleic acid encoding a chimeric protein that is at least 90% or at least 95% identical to SEQ ID NO: 1, SEQ ID NO:2, SEQ ID NO:3, or SEQ ID NO:4. In yet another related embodiment, the disclosure provides a recombinant nucleic acid encoding a chimeric protein of SEQ ID NO: 9, 10, 11, or 12. In yet another related embodiment, the disclosure provides a recombinant nucleic acid encoding a chimeric protein that is at least 80%, at least 85%, at least 90% or at least 95% identical to any of SEQ ID NOs: 9, 10, 11, or 12. In yet another embodiment, the disclosure provides a vector comprising a promoter operatively linked to a nucleic acid encoding any of the chimeric proteins described in the preceding paragraphs in the Summary.

In another embodiment, the disclosure provides an engineered cyanobacterium comprising any of the chimeric proteins enabling hydrocarbon efflux described in the preceding paragraphs in this Summary, or any of the recombinant nucleic acids described in the preceding paragraphs in this Summary. In a related embodiment, the disclosure provides such an engineered cyanobacterium, wherein said cyanobacterium further comprising one or more recombinant genes encoding an Aar enzyme, an Adm enzyme, or both enzymes. In a related embodiment, the engineered cyanobacterium is an engineered Synechococcus species. In another related embodiment, the engineered cyanobacterium is a thermophile.

In another embodiment, the disclosure provides a chimeric integral plasma membrane protein (CIPMP) for facilitating hydrocarbon efflux in a target bacterium, wherein said CIPMP comprises, at its N-terminus, a pseudo leader sequence, wherein said pseudo leader sequence is covalently fused to a heterologous IPMP, and wherein said pseudo leader sequence comprises one, two, three or four alpha helices and wherein the N-terminus of said CIPMP is in the cytoplasm when expressed in said target bacterium. In a related embodiment, wherein the IPMP, in its native state, has its N-terminus in the cytoplasm, the pseudo leader sequence consists of two transmembrane alpha helices and a periplasmic loop, or four transmembrane helices, two periplasmic loops, and a cytoplasmic loop. In a related embodiment, wherein the IPMP, in its native state, has its N-terminus in the periplasm, the pseudo leader sequence consists of one transmembrane alpha helices, or three transmembrane helices, two periplasmic loops and a cytoplasmic loop.

In another embodiment, the disclosure provides a method for producing hydrocarbons, comprising (i) culturing an engineered cyanobacterium described in the preceding paragraph in a culture medium, and (ii) exposing said engineered photosynthetic microorganism to light and carbon dioxide, wherein said exposure results in the conversion of said carbon dioxide by said engineered cyanobacterium into n-alkanes, wherein said n-alkanes are effluxed into said culture medium in an amount greater than that secreted by an otherwise identical cyanobacterium, cultured under identical conditions, but lacking any of the chimeric proteins or any of recombinant nucleic acids encoding the chimeric efflux proteins described above.

Various embodiments of the disclosure disclosed herein are further described in the Figures, Description, Examples, and Claims.

### FIGURES

FIG. 1: Design of pseudo-leader sequences for an Nᵢₙ heterologous integral plasma membrane protein (upper panel) and for an Nₒᵤₜ heterologous integral plasma membrane protein (lower panel). Transmembrane α helices are represented as rectangles (white for Nᵢₙ, and grey for Nₒᵤₜ, HIPMPs). The left column schematizes the native topology of the HIPMP, the right column the native topology of the non-JCC138 cyanobacterial PM protein from which the PLS is derived, and the center column the intended topology of the HIPMP bearing an N-terminal PLS fusion sequence when expressed in the target cyanobacterial host (diagonally hatched for Nᵢₙ and cross hatched for Nₒᵤₜ HIPMPs). HIPMP, Heterologous (with respect to JCC138) Integral Plasma Membrane Protein; PLS, pseudo-leader sequence; Nᵢₙ, integral membrane protein whose N-terminus resides inside the cytosol; Nₒᵤₜ, integral membrane protein whose N-terminus resides inside the periplasm. Ovals represent globular, soluble domains in either the cytoplasm or periplasm.

### DETAILED DESCRIPTION

Unless otherwise defined herein or in the above-mentioned utility applications, e.g., U.S. patent application Ser. No. 12/833,821, filed Jul. 9, 2010, scientific and technical terms used in connection with the present disclosure shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art.

Cyanobacteria contain not only a plasma membrane (PM) like non-photosynthetic prokaryotic hosts (as well as an outer membrane like their Gram-negative non-photosynthetic counterparts), but also, typically, an intracellular thylakoid membrane (TM) system that serves as the site for photosynthetic electron transfer and proton pumping. Given that both the plasma membrane and thylakoid membrane are typically loaded with proteins, both integral and peripheral, and, further, that a significant fraction of experimentally detected membrane proteins, both integral and peripheral, appear to be uniquely localized in each membrane, the question arises as to how differential localization of membrane proteins between the PM and TM is achieved in cyanobacteria (Rajalahti T et al. (2007) J Proteome Res 6:2420-2434). This question is of relevance to cyanobacterial metabolic engineering because certain heterologous enzymatic functions that may be desirable to engineer into said photosynthetic hosts are encoded by heterologous integral plasma membrane proteins (HIPMPs), both prokaryotic and eukaryotic in origin, that must be targeted to the plasma membrane of the cyanobacterial host in order to function as desired. The HIPMPs of interest in this respect comprise proteins that mediate transport, typically efflux, of substrates across the cyanobacterial plasma membrane. HIPMPs of particular interest correspond to the integral plasma membrane subunits, YbhS and YbhR, of a putative ATP-binding cassette (ABC) hydrocarbon efflux pump system from E. coli.

The methods described herein can be extended to integral membrane proteins that are not HIPMPs, i.e., proteins that are derived from membranes other than the plasma membrane. Such alternative membranes include: the thylakoid membrane, the endoplasmic reticulum membrane, the chloroplast inner membrane, and the mitochondrial inner membrane.

In one embodiment, the disclosure provides methods for designing a protein comprising a pseudo-leader sequence (PLS) of defined sequence fused to the N-terminus of an HIPMP of interest, wherein the resulting chimeric protein is expressed in a cyanobacterial host cell, e.g., JCC138 (Synechocystis sp. PCC 7002) or an engineered derivative thereof. The expression of the chimeric protein will increase the amount of hydrocarbon products of interest (e.g., alkanes, alkenes, alkyl alkanoates, etc.) exported from the cyanobacterial host cell. The PLS encodes a contiguous polypeptide sub-fragment of a protein from a different thylakoid-membrane-containing cyanobacterial host, e.g., JCC160 (Synechococcus sp. PCC 6803), that localizes as uniquely as possible to the plasma membrane of that host. The mechanism that this non-JCC138 host natively employs to effect the localization of the protein to the plasma membrane (rather than the thylakoid membrane) should be conserved in order for the localization to occur in the recipient host.

While PLSs are designed to ensure, or at least bias, the targeting of HIPMPs to the plasma membrane of the heterologous cyanobacterial host, they may not always be required. This is because sufficient levels of functional HIPMP may become embedded in the plasma membrane if the cyanobacterial host does, in fact, mechanistically recognize the protein as a native plasma membrane protein—even if some fraction of the protein is targeted to the thylakoid membrane or ends up in neither membrane (e.g., as inclusion bodies).

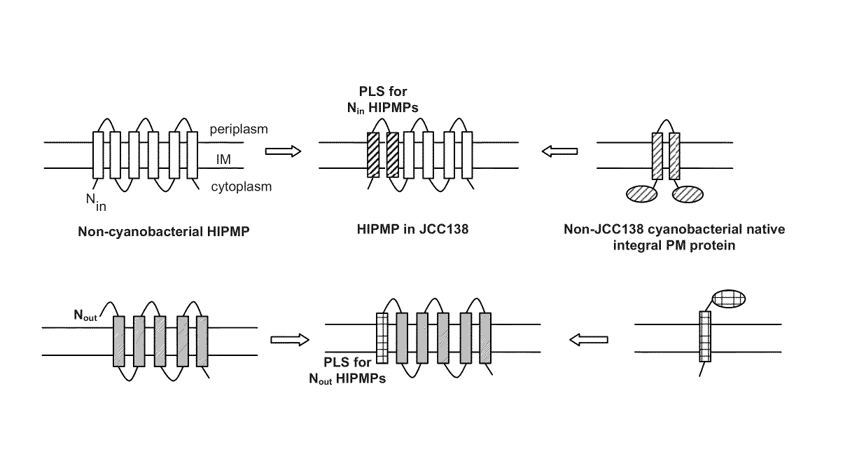

The strategy for identifying and designing functional PLSs is summarized in schematic form in FIG. 1.

For HIPMPs with cytoplasmic N-termini (Nᵢₙ), (i) the PLS is derived from a plasma-membrane-resident protein that is naturally anchored in the membrane of a different cyanobacterial species (i.e., different than the species into which the PLS will be functionally expressed) via two transmembrane α helices, and (ii) said plasma-membrane-resident protein naturally has its N-terminus within the cytoplasm and its C-terminus within the cytoplasm (Nᵢₙ/Cᵢₙ), spanning the plasma membrane via an in-to-out transmembrane α helix, followed by an (ideally short) periplasmic loop sequence, followed by an out-to-in transmembrane α helix. Correspondingly, for HIPMPs with periplasmic N-termini (Nₒᵤₜ), (i) the PLS is derived from a plasma-membrane-resident protein that is naturally anchored in the membrane of a different cyanobacterial species via one transmembrane α helix, and (ii) said plasma-membrane-resident protein naturally has its N-terminus within the cytoplasm and its C-terminus within the periplasm (Nᵢₙ/Cₒᵤₜ).

In a preferred embodiment, PLSs are derived from host proteins that have most of their mass in either the periplasmic and/or cytoplasmic spaces. In another preferred embodiment, said PLSs should contain only two α helices with Nᵢₙ/Cᵢₙ topology (FIG. 1, right column; for creating Nᵢₙ HIPMPs) and only one α helix with Nᵢₙ/Cₒᵤₜ topology (FIG. 1, right column; for creating Nₒᵤₜ HIPMPs). In a related embodiment, the potential for intermolecular homomultimerization among the transmembrane helices of the PLSs is minimized.

The terms “fused”, “fusion” or “fusing” used herein in the context of chimeric proteins refers to the joining of one functional protein or protein subunit (e.g., a pseudo-leader sequence) to another functional protein or protein subunit (e.g., an integral plasma membrane protein). Fusing can occur by any method which results in the covalent attachment of the C-terminus of one such protein molecule to the N-terminus of another. For example, one skilled in the art will recognize that fusing occurs when the two proteins to be fused are encoded by a recombinant nucleic acid under control of a promoter and expressed as a single structural gene in vivo or in vitro.

As used herein, the term “non-target” refers to a protein or nucleic acid that is native to a species that is different than the species that will be used to recombinantly express the protein or nucleic acid.

Alkanes, also known as paraffins, are chemical compounds that consist only of the elements carbon (C) and hydrogen (H) (i.e., hydrocarbons), wherein these atoms are linked together exclusively by single bonds (i.e., they are saturated compounds) without any cyclic structure. n-Alkanes are linear, i.e., unbranched, alkanes.

Genes encoding Aar or Adm enzymes are referred to herein as Aar genes (aar) or Adm genes (adm), respectively. Together, AAR and ADM enzymes function to synthesize n-alkanes from acyl-ACP molecules. As used herein, an Aar enzyme refers to an enzyme with the amino acid sequence of the SYNPCC7942_{—}1594 protein or a homolog thereof, wherein a SYNPCC7942_{—}1594 homolog is a protein whose BLAST alignment (i) covers >90% length of SYNPCC7942_{—}1594, (ii) covers >90% of the length of the matching protein, and (iii) has >50% identity with SYNPCC7942_{—}1594 (when optimally aligned using the parameters provided herein), and retains the functional activity of SYNPCC7942_{—}1594, i.e., the conversion of an acyl-ACP (acyl-acyl carrier protein) to an n-alkanal. An Adm enzyme refers to an enzyme with the amino acid sequence of the SYNPCC7942_{—}1593 protein or a homolog thereof, wherein a SYNPCC7942_{—}1593 homolog is defined as a protein whose amino acid sequence alignment (i) covers >90% length of SYNPCC7942_{—}1593, (ii) covers >90% of the length of the matching protein, and (iii) has >50% identity with SYNPCC7942_{—}1593 (when aligned using the preferred parameters provided herein), and retains the functional activity of SYNPCC7942_{—}1593, i.e., the conversion of an n-alkanal to an (n-1)-alkane. Exemplary Aar and Adm enzymes are listed in Table 1 and Table 2, respectively, of U.S. utility application Ser. No. 12/759,657, filed Apr. 13, 2010 (now U.S. Pat. No. 7,794,969), and U.S. utility application Ser. No. 12/833,821, filed Jul. 9, 2010. Other alkanal deformylative monooxygenase (“ADM”) activities are described in U.S. patent application Ser. No. 12/620,328, filed Nov. 17, 2009. Applicants note that the ADM enzyme described herein was referred to in earlier related application as “alkanal decarboxylative monooxygenase”. To be clear, the proteins are identical; only the name is changed in this application as additional details about the mechanism of the reaction catalyzed by the enzyme has become known.

Preferred parameters for BLASTp are: Expectation value: 10 (default); Filter: none; Cost to open a gap: 11 (default); Cost to extend a gap: 1 (default); Maximum alignments: 100 (default); Word size: 11 (default); No. of descriptions: 100 (default); Penalty Matrix: BLOWSUM62.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

One skilled in the art will also recognize, in light of the teachings herein, that the methods and compositions described herein for use in particular organisms, e.g., cyanobacteria, are also applicable other organisms, e.g., gram-negative bacteria such as E. coli. For example, a chimeric integral plasma membrane protein for facilitating alkane efflux in E. coli could be designed by fusing a pseudo leader sequence derived from E. coli or a related bacterium to a heterologous integral plasma membrane protein.

The following terms, unless otherwise indicated, shall be understood to have the following meanings

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native intemucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

As used herein, an “isolated” organic molecule (e.g., an alkane, alkene, or alkanal) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present disclosure may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, intemucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

The term “deletion” refers to the removal of one or more nucleotides from a nucleic acid molecule or one or more amino acids from a protein, the regions on either side being joined together.

The term “knock out” refers to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present disclosure have particular utility. The heterologous polypeptide included within the fusion protein of the present disclosure is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′).sub.2, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications, (Marasco, ed., Springer-Verlag New York, Inc., 1998), the disclosure of which is incorporated herein by reference in its entirety).

As used herein, antibodies can be produced by any known technique, including harvest from cell culture of native B lymphocytes, harvest from culture of hybridomas, recombinant expression systems and phage display.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present disclosure may be used to produce an equivalent effect and are therefore envisioned to be part of the present disclosure.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred embodiment, a mutein exhibits at least 95% sequence identity, even more preferably 98%, even more preferably 99% and even more preferably 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-,α-disubstituted amino acids, N-alkyl amino acids, and other unconventional amino acids may also be suitable components for polypeptides of the present disclosure. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

“Percent dry cell weight” refers to a measurement of hydrocarbon production obtained as follows: a defined volume of culture is centrifuged to pellet the cells. Cells are washed then dewetted by at least one cycle of microcentrifugation and aspiration. Cell pellets are lyophilized overnight, and the tube containing the dry cell mass is weighed again such that the mass of the cell pellet can be calculated within ±0.1 mg. At the same time cells are processed for dry cell weight determination, a second sample of the culture in question is harvested, washed, and dewetted. The resulting cell pellet, corresponding to 1-3 mg of dry cell weight, is then extracted by vortexing in approximately 1 ml acetone plus butylated hydroxytolune (BHT) as antioxidant and an internal standard, e.g., n-eicosane. Cell debris is then pelleted by centrifugation and the supernatant (extractant) is taken for analysis by GC. For accurate quantitation of n-alkanes, flame ionization detection (FID) is used as opposed to MS total ion count. n-Alkane concentrations in the biological extracts are calculated using calibration relationships between GC-FID peak area and known concentrations of authentic n-alkane standards. Knowing the volume of the extractant, the resulting concentrations of the n-alkane species in the extracant, and the dry cell weight of the cell pellet extracted, the percentage of dry cell weight that comprised n-alkanes can be determined.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

“Carbon-based Products of Interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, Docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

Biofuel: A biofuel refers to any fuel that derives from a biological source. Biofuel can refer to one or more hydrocarbons, one or more alcohols, one or more fatty esters or a mixture thereof.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Throughout this specification and claims, the word “comprise” or variations such as “comprises” or “comprising”, will be understood to imply the inclusion of a stated integer or group of integers but not the exclusion of any other integer or group of integers.

In another embodiment, the nucleic acid molecule of the present disclosure encodes a polypeptide having the amino acid sequence of SEQ ID NO:1, 2, 3, 4, 9, 10, 11 or 12. Preferably, the nucleic acid molecule of the present disclosure encodes a polypeptide sequence of at least 50%, 60, 70%, 80%, 85%, 90% or 95% identity to SEQ ID NO:1, 2, 3, 4, 9, 10, 11 or 12 and the identity can even more preferably be 96%, 97%, 98%, 99%, 99.9% or even higher.

The present disclosure also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The nucleic acid sequence fragments of the present disclosure display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hydridization to target sequences can be detected and optionally quantified. One of skill in the art will appreciate that the nucleic acid fragments of the present disclosure may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosure of each of which is incorporated herein by reference in its entirety.

As is well known in the art, enzyme activities can be measured in various ways. For example, the pyrophosphorolysis of OMP may be followed spectroscopically (Grubmeyer et al., (1993) J. Biol. Chem. 268:20299-20304). Alternatively, the activity of the enzyme can be followed using chromatographic techniques, such as by high performance liquid chromatography (Chung and Sloan, (1986) J. Chromatogr. 371:71-81). As another alternative the activity can be indirectly measured by determining the levels of product made from the enzyme activity. These levels can be measured with techniques including aqueous chloroform/methanol extraction as known and described in the art (Cf. M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y.: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present disclosure. Other methods and techniques may also be suitable for the measurement of enzyme activity, as would be known by one of skill in the art.

Also provided by the present disclosure are vectors, including expression vectors, which comprise the above nucleic acid molecules of the present disclosure, as described further herein. In a first embodiment, the vectors include the isolated nucleic acid molecules described above. In an alternative embodiment, the vectors of the present disclosure include the above-described nucleic acid molecules operably linked to one or more expression control sequences. The vectors of the instant disclosure may thus be used to express an Aar and/or Adm polypeptide contributing to n-alkane producing activity by a host cell, and/or a chimeric efflux protein for effluxing n-alkanes and other hydrocarbons out of the cell.

In another aspect of the present disclosure, host cells transformed with the nucleic acid molecules or vectors of the present disclosure, and descendants thereof, are provided. In some embodiments of the present disclosure, these cells carry the nucleic acid sequences of the present disclosure on vectors, which may but need not be freely replicating vectors. In other embodiments of the present disclosure, the nucleic acids have been integrated into the genome of the host cells.

In a preferred embodiment, the host cell comprises one or more AAR or ADM encoding nucleic acids which express AAR or ADM in the host cell.

In an alternative embodiment, the host cells of the present disclosure can be mutated by recombination with a disruption, deletion or mutation of the isolated nucleic acid of the present disclosure so that the activity of the AAR and/or ADM protein(s) in the host cell is reduced or eliminated compared to a host cell lacking the mutation.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure-tolerant organisms include piezophiles, which tolerate pressure of 130 MPa. Weight-tolerant organisms include barophiles. Hypergravity (e.g., >1 g) and hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt-tolerant organisms include halophiles (e.g., 2-5 M NaCl) Halobacteriacea and Dunaliella salina. pH-tolerant organisms include alkaliphiles such as Natronobacterium, Bacillus firmus OF4, Spirulina spp. (e.g., pH >9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas-tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New York: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium. A partial list of cyanobacteria that can be engineered to express the recombinant described herein include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochiorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospermum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera:

Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis,

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

Preferred organisms for the manufacture of n-alkanes according to the methods disclosed herein include: Arabidopsis thaliana, Panicum virgatum, Miscanthus giganteus, and Zea mays (plants); Botryococcus braunii, Chlamydomonas reinhardtii and Dunaliela salina (algae); Synechococcus sp PCC 7002, Synechococcus sp. PCC 7942, Synechocystis sp. PCC 6803, Thermosynechococcus elongatus BP-1 (cyanobacteria); Chlorobium tepidum (green sulfur bacteria), Chloroflexus auranticus (green non-sulfur bacteria); Chromatium tepidum and Chromatium vinosum (purple sulfur bacteria); Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A suitable organism for selecting or engineering is autotrophic fixation of CO₂ to products. This would cover photosynthesis and methanogenesis. Acetogenesis, encompassing the three types of CO₂ fixation; Calvin cycle, acetyl-CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. See, e.g., Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

For producing n-alkanes via the recombinant expression of Aar and/or Adm enzymes, an engineered cyanobacterium, e.g., a Synechococcus or Thermosynechococcus species, is preferred. Other preferred organisms include Synechocystis, Klebsiella oxytoca, Escherichia coli or Saccharomyces cerevisiae. Other prokaryotic, archaeal and eukaryotic host cells are also encompassed within the scope of the present disclosure.

In various embodiments of the disclosure, desired hydrocarbons and/or alcohols of certain chain length or a mixture thereof can be produced. In certain aspects, the host cell produces at least one of the following carbon-based products of interest: 1-dodecanol, 1-tetradecanol, 1-pentadecanol, n-tridecane, n-tetradecane, 15:1 n-pentadecane, n-pentadecane, 16:1 n-hexadecene, n-hexadecane, 17:1 n-heptadecene, n-heptadecane, 16:1 n-hexadecen-ol, n-hexadecan-1-ol and n-octadecen-1-ol, as shown in the Examples herein. In other aspects, the carbon chain length ranges from C₁₀ to C₂₀. Accordingly, the disclosure provides production of various chain lengths of alkanes, alkenes and alkanols suitable for use as fuels & chemicals.

In preferred aspects, the methods of the present disclosure include culturing host cells for direct product secretion for easy recovery without the need to extract biomass. These carbon-based products of interest are secreted directly into the medium. Since the disclosure enables production of various defined chain length of hydrocarbons and alcohols, the secreted products are easily recovered or separated. The products of the disclosure, therefore, can be used directly or used with minimal processing.

In various embodiments, compositions produced by the methods of the disclosure are used as fuels. Such fuels comply with ASTM standards, for instance, standard specifications for diesel fuel oils D 975-09b, and Jet A, Jet A-1 and Jet B as specified in ASTM Specification D. 1655-68. Fuel compositions may require blending of several products to produce a uniform product. The blending process is relatively straightforward, but the determination of the amount of each component to include in a blend is much more difficult. Fuel compositions may, therefore, include aromatic and/or branched hydrocarbons, for instance, 75% saturated and 25% aromatic, wherein some of the saturated hydrocarbons are branched and some are cyclic. Preferably, the methods of the disclosure produce an array of hydrocarbons, such as C₁₃-C₁₇ or C₁₀-C₁₅ to alter cloud point. Furthermore, the compositions may comprise fuel additives, which are used to enhance the performance of a fuel or engine. For example, fuel additives can be used to alter the freezing/gelling point, cloud point, lubricity, viscosity, oxidative stability, ignition quality, octane level, and flash point. Fuels compositions may also comprise, among others, antioxidants, static dissipater, corrosion inhibitor, icing inhibitor, biocide, metal deactivator and thermal stability improver.

In addition to many environmental advantages of the disclosure such as CO₂ conversion and renewable source, other advantages of the fuel compositions disclosed herein include low sulfur content, low emissions, being free or substantially free of alcohol and having high cetane number.

In another aspect, the present disclosure provides isolated antibodies, including fragments and derivatives thereof that bind specifically to the isolated polypeptides and polypeptide fragments of the present disclosure or to one or more of the polypeptides encoded by the isolated nucleic acids of the present disclosure. The antibodies of the present disclosure may be specific for linear epitopes, discontinuous epitopes or conformational epitopes of such polypeptides or polypeptide fragments, either as present on the polypeptide in its native conformation or, in some cases, as present on the polypeptides as denatured, as, e.g., by solubilization in SDS. Among the useful antibody fragments provided by the instant disclosure are Fab, Fab′, Fv, F(ab′)₂, and single chain Fv fragments.

The following examples are for illustrative purposes and are not intended to limit the scope of the present disclosure.

### EXAMPLES

### Example 1

### Method for Identifying and Designing a Pseudo Leader Sequence

This Example describes the application of the methodology described above to the design of PLSs suitable for expressing a HIPMP in JCC138 (Synechococcus sp. PCC 7002). JCC160 (Synechocystis sp. PCC 6038) was chosen as the source for the PLSs.

Initially, proteins were identified as having been experimentally demonstrated to be integral membrane PM proteins in JCC160 with only one or two transmembrane α helices. Thirty proteins were identified based on publications in the field (see Table 2 of Huang F. et al. (2002) Mol Cell Proteomics 1:956-966, Table 1 of Huang F. et al. (2006) Proteomics 6:910-920, and Table 2 of Pisareva T. et al. (2007) FEBS 274:791-804).

To investigate further the potential for each protein to contain a PLS, the membrane topology of each was predicted using two different programs: Philius (described in Reynolds S M et al. (2008) PLoS Comput Biol 4:e1000213) and TOPCONS (described in Bernsel A et al. (2009) Nucleic Acids Res 37:W465-468). As an added specificity measure, the potential for the N-terminal region of each protein to encode a cleavable signal sequence characteristic of either signal peptidase I (LepB) or signal peptidase II (LspA), was also evaluated using the program LipoP (described in Juncker A S et al. (2003) Protein Sci 12:1652-1662). In addition, the closest homolog of each JCC160 protein was identified in JCC138 using pBLAST; each such JCC138 homolog was itself submitted to Philius, TOPCONS, and LipoP. The results of these analyses are summarized in Table 1A and Table 1B.

TABLE 1A
Table 1A Experimentally verified integral PM proteins of JCC160 based on literature analysis
(see text). The full sequence of each JCC160 protein was subjected to two different transmembrane
topology prediction servers: Philius (http://www.yeastrc.org/philius/pages/philius/runPhilius.jsp)
and TOPCONS (http://topcons.net), as well as to the LipoP server for prediction of lipoproteins
and signal peptides in Gram-negative bacteria (http://www.cbs.dtu.dk/services/LipoP). The best-
hit homolog of each JCC160 protein was determined by querying the JCC138 proteome using pBLAST
(http://www.ncbi.nlm.nih.gov//genomes/geblast.cgi?gi=22070).

| | | | | | JCC160 LipoP | JCC160 | JCC160 |
|---|---|---|---|---|---|---|---|
| | JCC160 | | | | prediction | N-term. | # transmem- |
| JCC160 | length | | JCC160 | JCC160 TOPCONS | (if predicted, −1/+1 | designation | brane α |
| Locus | (aa) | Annotation | Philius prediction | prediction | cleavage junction) | (NR) | helices(NR) |
| sll1323 | 143 | ATP synthase subunit b′, | Nout, 7-27 | Nout, 7-27 | nd | N-out | 1 |
| | | AtpF | | | | | |
| sll1324 | 179 | ATP synthase subunit b, | Nout, 29-47 | Nout, 25-45 | nd | N-out | 1 |
| | | AtpF | | | | | |
| sll1665 | 589 | Uncharacterized protein, | Signal peptide, 1-19 | Nout, 6-26 | nd | N-out | 1 |
| | | FtsY homolog | | | | | |
| sll0034 | 258 | VanY putative D-alanyl-D- | Nin, 38-59 | Nin, 38-58 | No ss predicted | N-in | 1 |
| | | alanine carboxypeptidase | | | | | |
| sll0606 | 476 | periplasmic ligand-binding | Nin, 21-41 | Nin, 21-41 | No ss predicted | N-in | 1 |
| | | domain | | | | | |
| sll1053 | 520 | RND family efflux | Nin, 40-58 | Nin, 40-60 | No ss predicted | N-in | 1 |
| | | transporter MFP subunit | | | | | |
| sll1181 | 583 | HlyD family of secretion | Nin, 62-84 | Nout, 64-84 | No ss predicted | N-in | 1 |
| | | proteins | | | | | |
| sll1405 | 142 | ExbD-like protein | Nin, 21-43 | Nin, 21-41 | No ss predicted | N-in | 1 |
| sll1694 | 168 | General secretion pathway | Nin, 21-42 | Nin, 21-41 | No ss predicted | N-in | 1 |
| | | protein G, hofG | | | | | |
| slr0013 | 175 | Uncharacterized protein | Nin, 11-35 | Nin, 15-35 | No ss predicted | N-in | 1 |
| slr1106 | 282 | Prohibitin, phb | Nin, 10-32 | Nin, 12-32 | No ss predicted | N-in | 1 |
| slr1377 | 218 | Signal peptidase of plasma | Globular | Nin, 21-41 | No ss predicted | N-in | 1 |
| | | membane, LepB2 | | | | | |
| slr1721 | 492 | Uncharacterized protein | Nin, 17-38 | Nin, 21-41 | No ss predicted | N-in | 1 |
| slr1730 | 190 | Potassium-transporting | Nin, 12-35 | Nin, 11-31 | AIVLV₂₉\| I₃₀GQLV | N-in | 1 |
| | | ATPase C chain | | | | | |
| sll0041 | 1000 | Methyl-accepting | Nout, 200-221, 247-266 | Nin, 200-220, 247-267 | No ss predicted | N-in | 2 |
| | | chemotaxis | | | | | |
| | | protein, PisJ1 | | | | | |
| sll1294 | 953 | Methyl-accepting | Nin, 218-241, 528-550 | Nin, 218-238, 530-550 | No ss predicted | N-in | 2 |
| | | chemotaxis like | | | | | |
| | | protein pilJ | | | | | |
| slr0483 | 149 | Uncharacterized protein | Nin, 77-97, 105-127 | Nin, 77-97, 105-125 | No ss predicted | N-in | 2 |
| slr0875 | 145 | Large-conduct. | Nin, 21-51, 72-94 | Nin, 25-45, 74-94 | No ss predicted | N-in | 2 |
| | | mechanosensitive | | | | | |
| | | channel, MscL | | | | | |
| slr1044 | 869 | Methyl-accepting | Nout, 382-406, 447-467 | Nin, 382-402, 447-467 | No ss predicted | N-in | 2 |
| | | chemotaxis | | | | | |
| | | protein, Ctr1 | | | | | |
| slr1943 | 331 | Uncharacterized | Nin, 247-269, 281-306 | Nin, 247-267, 283-303 | No ss predicted | N-in | 2 |
| | | glycosyltransferase | | | | | |
| sll0923 | 756 | Exopolysaccharide export | Nin, 41-57 | Nin, 42-62, 454-474 | nd | N-in | ? |
| | | protein, EpsB | | | | | |
| sll0813 | 300 | Cytochrome c oxidase | Nin, 7-25, 48-70, 91-110 | Nin, 6-26, 46-66, 90-110 | nd | N-in | ? |
| | | subunit 2, CtaC | | | | | |
| sll1021 | 673 | Band-7 flotillin | Nin, 60-82 | Nout, 60-80 | nd | ? | 1 |
| sll1484 | 524 | Type II NADH | Nout, 484-504 | Nin, 485-505 | nd | ? | 1 |
| | | dehydrogenase | | | | | |
| slr1275 | 284 | Fimbrial assembly protein | Nin, 38-61 | Nout, 38-58 | nd | ? | 1 |
| | | (PilN) family | | | | | |
| slr1276 | 275 | Uncharacterized protein | Nin, 37-61 | Nout, 37-57 | nd | ? | 1 |
| slr1390 | 665 | Cell division protease | Signal peptide, 1-40 | Nin, 21-41, 160-180 | nd | ? | ? |
| | | ftsH homolog 2 | | | | | |
| slr1604 | 616 | Cell division protease | Signal peptide, 1-27 | Nin, 10-30, 109-129 | nd | ? | ? |
| | | ftsH homolog 4 | | | | | |
| slr6071 | 730 | Uncharacterized protein | Nin, 11-31 | Nout, 11-31 | nd | ? | 1 |
| slr1768 | 298 | Prohibitin | Signal peptide, 1-53 | Nout, 2-22, 35-55 | nd | ? | ? |

na, not applicable; nd, not done, because either the N-terminus was non-cytoplasmic and/or because the number of predicted transmembrane helices and absence of cleavable signal sequence was inconsistent across the Philius, TOPCONS, and LipoP predictions; NR, as assessed by Nikos Reppas, taking into account all three prediction data types; ss, signal sequence; Nin, cytosolic N-terminus; Nout, periplasmic N-terminus. Rows in bold correspond to the final JCC160 proteins selected for designing Nᵢₙ and Nₒᵤₜ PLSs.

TABLE 1B

| | | | | | JCC138 LipoP | JCC160/JCC138 |
|---|---|---|---|---|---|---|
| | | JCC138 | | | prediction | conserved |
| JCC160 | JCC138 best-hit | length | JCC138 | JCC138 TOPCONS | (if predicted, −1/+1 | transmembrane |
| Locus | homolog | (aa) | Philius prediction | prediction | cleavage junction) | α helical region? |
| sll1323 | SYNPCC7002_A0737 | 161 | nd | nd | nd | nd |
| sll1324 | SYNPCC7002_A0736 | 175 | nd | nd | nd | nd |
| sll1665 | No match | na | nd | nd | nd | na |
| sll0034 | SYNPCC7002_{—}A2145 | 261 | Nin, 40-57 | Nin, 38-58 | No ss predicted | No |
| sll0606 | SYNPCC7002_A2745 | 466 | Nin, 8-29 | Nin, 9-29 | LGLLG₂₂\| A₂₃GGWW | Yes |
| sll1053 | SYNPCC7002_{—}A1574 | 420 | Signal peptide 1-34 | Nin, 8-28 | VSLSG₂₆\| C₂₇GGPP | No |
| sll1181 | SYNPCC7002_G0070 | 532 | Signal peptide 1-28 | Nin, 12-32 | TVAWA₂₈\| A₂₉IAKI | Yes |
| sll1405 | SYNPCC7002_G0136 | 140 | Nin, 12-36 | Nin, 16-36 | No ss predicted | Yes |
| sll1694 | SYNPCC7002_A2803 | 175 | Nin, 21-46 | Nin, 21-41 | No ss predicted | Yes |
| slr0013 | SYNPCC7002_A0687 | 177 | Nin, 14-35 | Nin, 16-36 | No ss predicted | Yes |
| slr1106 | SYNPCC7002_A2606 | 280 | Nin, 10-32 | Nin, 12-32 | LNAFV₃₁\| I₃₂INPG | No |
| slr1377 | SYNPCC7002_A1435 | 208 | Globular | Nin, 29-49 | No ss predicted | Yes |
| slr1721 | SYNPCC7002_A0189 | 488 | Signal peptide 1-36 | Nin, 10-30 | No ss predicted | No |
| slr1730 | SYNPCC7002_G0055 | 190 | Nin, 12-35 | Nin, 10-30 | No ss predicted | Yes |
| sll0041 | SYNPCC7002_{—}A0048 | 1490 | Globular | Globular | No ss predicted | No |
| sll1294 | SYNPCC7002_A2599 | 1014 | Nin, 266-289, 589-611 | Nin, 246-266, 571-591; | No ss predicted | Yes |
| | | | | some 266-286, 591-611 | | |
| slr0483 | SYNPCC7002_A2448 | 135 | Nin, 62-83, 90-112 | Nin, 62-82, 90-110 | No ss predicted | Yes |
| slr0875 | SYNPCC7002_A2462 | 145 | Nin, 20-37, 75-93 | Nin, 20-40, 75-95 | No ss predicted | Yes |
| slr1044 | SYNPCC7002_{—}A1372 | 902 | Nout, 437-459, 477-497 | Nin, 437-457, 477-497 | No ss predicted | No |
| slr1943 | SYNPCC7002_A0766 | 313 | Nin, 229-253, 261-284 | Nin, 229-249, 265-285 | No ss predicted | Yes |
| sll0923 | SYNPCC7002_A1500 | 756 | nd | nd | nd | nd |
| sll0813 | SYNPCC7002_A0727 | 297 | nd | nd | nd | nd |
| sll1021 | SYNPCC7002_A2510 | 657 | nd | nd | nd | nd |
| sll1484 | SYNPCC7002_A2120 | 390 | nd | nd | nd | nd |
| slr1275 | SYNPCC7002_A0502 | 243 | nd | nd | nd | nd |
| slr1276 | SYNPCC7002_A0503 | 268 | nd | nd | nd | nd |
| slr1390 | SYNPCC7002_A0349 | 628 | nd | nd | nd | nd |
| slr1604 | SYNPCC7002_A0040 | 620 | nd | nd | nd | nd |
| slr6071 | SYNPCC7002_A1674 | 456 | nd | nd | nd | nd |
| slr1768 | SYNPCC7002_A2606 | 280 | nd | nd | nd | nd |

Table 1B See legend to Table 1A.

Highlighted in Table 1A and Table 1B are the two JCC160 proteins containing the top-candidate Nₒᵤₜ PLSs, sll0034 and sll1053, as well as the JCC160 proteins containing the top-candidate Nᵢₙ PLSs, sll0041 and slr1044. Their candidacy is based primarily on two criteria: (1) the predicted transmembrane topology of the proteins is consistent with their annotation and likely biological function, and (2) there is minimal sequence conservation between the putative transmembrane α helical regions of the JCC160 PLS-containing protein and the corresponding best-BLAST-hit JCC138 homolog (see last column of Table 1B above).

In terms of the predicted transmembrane topology, (i) the C-terminal region of sll0034 encodes a VanY-type D-alanyl-D-alanine carboxypeptidase, an enzyme involved in peptidoglycan synthesis; it is therefore reasonable to assume that the protein protrudes into the periplasmic space, (ii) sll1053 encodes a membrane fusion protein, a class of proteins that is known to function in the periplasmic space; many such proteins are known to be tethered to the inner membrane via either acyl tails or a single transmembrane α helix, (iii) the domain structure and function of sll0041, encoding PisJ1, has been discussed in Yoshihara S et al. (2000) Plant Cell Physiol 41:1299-1304; this protein is a homolog of the Che proteins involved in flagellar switching for chemotaxis that are known to be PM-tethered via two transmembrane helices in bacteria like E. coli; significantly, the putative periplasmic region between the two transmembrane helices of sll0041 is small and therefore unlikely to be functional significance, and, indeed, the sensor domain of this phototaxis protein appears to be a phytochrome-like photoreceptor in the cytosol, and (iv) the domain structure and function of slr1041, encoding Ctr1, has been discussed in Chung Y-H et al. (2001) FEBS Lett 492:33-38; this protein is also a Che protein that is involved in gliding motility and biogenesis of thick pili; it therefore consistent that this protein should be localized in the plasma membrane; significantly, its periplasmic region is also small and is therefore likely to be of little functional importance.

With regard to the sequence conservation between the putative transmembrane α helical regions of the JCC160 PLS-containing protein and the corresponding best-BLAST-hit JCC138 homolog, the alignment of each JCC160 protein and its best-BLAST-hit JCC138 homolog was manually examined to determine the degree of sequence conservation of the consistently predicted transmembrane α helical regions in each, relative to the rest of the protein. An assessment of the degree of transmembrane α helical sequence conservation was determined by visual inspection. Having selected the aforementioned JCC160 PM proteins, the corresponding predicted transmembrane helical regions and flanking sequences were extracted as the final two Nₒᵤₜ, and two Nᵢₙ, PLSs (Table 2). For the former, the N-terminal flanking sequence was taken from the native start codon as the single transmembrane helix was near the native N-terminus of the protein, whereas for the latter, it was taken from the middle of the protein because the putative transmembrane helical region is situated there (Table 1). The corresponding native JCC160 DNA sequences coding for these four PLSs are tabulated in Table 3. In another embodiment, codon optimization, e.g., via DNA2.0, could be used to increase expression in JCC138 (or in an engineered hydrocarbon-producing strain derived from JCC138).

TABLE 2
Table 2 Selected pseudo-leader sequences for N-terminal fusion to HIPMPs to be
expressed in JCC138 (or another heterologous target cyanobacterium of interest).

| Pseudo-leader | PLS | PLS length | Peptide sequence to replace the start-codon amino acid |
|---|---|---|---|
| sequence (PLS) | type | (aa) | of the native HIPMP to enable targeting to the PM |
| sll0034_Nout_PLS | Nₒᵤₜ | 73 | mgkkpkssfnvnqddipevirdnpagspqsqplspipLKLIAAGLGIII |
| | | | LALLTLLALwprpepapevvteps |
| | | | SEQ ID NO: 1 |
| sll1053_Nout_PLS | Nₒᵤₜ | 69 | mteppvlhetssesekeqsigkqnlsfqpipqaskpgkrLWLVVGALLL |
| | | | LGGGGYWWFQSrsggppgga |
| | | | SEQ ID NO: 2 |
| sll0041_Nin_PLS | Nᵢₙ | 97 | MqaptqsgglslrnkAVLIALLIGLIPAGVIGGLNLSsvdrlpvpqteq |
| | | | qvkdsttkqirdqILIGLLVTAVGAAFVAYWMVGentkaqtalalkak |
| | | | SEQ ID NO: 3 |
| slr1044_Nin_PLS | Nᵢₙ | 116 | MflgwftnaslfrkqIYMAIASGVFSGFAVLVLGSIVGLGgtpkdvpap |
| | | | sgettteapaegapaegqapsqtpeeepgkpSLLNLAFLTAIATAIGVF |
| | | | LINrllmqqiksiiddlq |
| | | | SEQ ID NO: 4 |

Predicted transmembrane α helical region(s) are capitalized and underlined. In the case of sll0041_Nin_PLS and slr1044_Nin_PLS, a non-native start-codon methionine must be inserted (capitalized and bold). For all PLSs, 9-15 native amino acids are included after the C-terminus of the last putative helical region to serve as a spacer between said helix and the native N-terminal +2 residue of the HIPMP.

TABLE 3
Table 3 Native JCC160 DNA sequences encoding the PLSs in Table 2.

| Pseudo-leader sequence | |
|---|---|
| (PLS) ID | DNA coding sequence |
| sll0034_Nout_PLS | ATGGGTAAAAAACCAAAATCTTCCTTTAACGTCAACCAGGATGACATCCCGGAAGTAAT |
| | TCGGGACAATCCAGCGGGGTCGCCCCAGTCCCAACCCTTGTCCCCCATACCGCTGAAAT |
| | TAATCGCGGCCGGCTTGGGGATAATTATTCTGGCTTTATTGACGTTGTTGGCCCTGTGG |
| | CCCAGGCCCGAGCCCGCACCGGAAGTGGTGACAGAACCAAGT |
| | SEQ ID NO: 5 |
| sll1053_Nout_PLS | ATGACTGAGCCACCCGTACTGCACGAAACTTCTTCGGAATCGGAAAAGGAACAAAGTAT |
| | TGGTAAACAGAATTTGTCCTTTCAACCCATTCCCCAAGCCTCCAAACCAGGCAAACGAC |
| | TCTGGCTAGTGGTGGGAGCCTTACTGTTGCTAGGAGGTGGTGGCTACTGGTGGTTTCAG |
| | TCCCGTTCCGGCGGCCCTCCCGGAGGGGCC |
| | SEQ ID NO: 6 |
| sll0041_Nin_PLS | ATGCAGGCACCTACCCAAAGTGGAGGACTTTCCCTCCGCAATAAAGCTGTGTTGATAGC |
| | CCTGTTAATTGGTTTGATCCCCGCTGGGGTGATTGGAGGGCTCAATCTCAGCAGTGTGG |
| | ACAGGCTTCCAGTGCCCCAAACGGAACAACAGGTCAAGGACTCCACCACTAAGCAAATC |
| | CGTGACCAAATTCTGATTGGGCTTTTGGTGACCGCGGTGGGGGCTGCCTTCGTTGCCTA |
| | CTGGATGGTGGGGGAAAATACCAAAGCTCAAACCGCCCTGGCCTTGAAAGCTAAG |
| | SEQ ID NO: 7 |
| slr1044_Nin_PLS | ATGTTTCTCGGTTGGTTCACCAATGCTTCCCTGTTTAGAAAGCAGATTTACATGGCGAT |
| | CGCCTCCGGTGTGTTTTCCGGGTTCGCGGTGTTGGTATTGGGAAGCATAGTGGGTTTAG |
| | GGGGAACTCCCAAAGACGTACCTGCTCCTTCAGGGGAAACCACCACCGAAGCTCCAGCA |
| | GAAGGTGCCCCCGCAGAGGGCCAGGCCCCTTCCCAGACCCCAGAGGAAGAACCGGGCAA |
| | ACCATCCCTCCTCAACCTCGCCTTCCTCACAGCCATAGCCACGGCGATCGGAGTCTTTC |
| | TGATCAATCGATTGCTGATGCAACAGATTAAGAGTATTATCGACGACCTGCAA |
| | SEQ ID NO: 8 |

### Example 2

### Detection of a Functionally Expressed HIPMP in a Cyanobacterium

The HIPMP to be expressed in JCC138 can detected by Western blotting, e.g., by virtue of a C-terminal epitope tag such as (His)₆ (SEQ ID NO: 13). Two different versions of said HIPMP are expressed, one corresponding to the native sequence, and the other to the same sequence N-terminally fused to the appropriate PLS (Nᵢₙ or Nₒᵤₜ, as required). Then the OM, PM, and TM fractions are fractionated. Each fraction is probed for the presence of either version of the tagged HIPMP. A higher fraction of membrane-associated tagged HIPMP is then found in the PM fraction of the PLS version than for the non-PLS version of the protein, and a functional assay for the PM-embedded HIPMP (e.g., increased yields of a secreted hydrocarbon of interest) indicates that the protein functions as expected.

The degree of alkane efflux into the medium can be assessed by extracting the raw cyanobacterial culture, or the cell-free spent medium from such a culture, in an organic solvent that phase-partitions when added to aqueous solutions, e.g., iso-octane. The concentration of alkane species of interest in the organic solvent following phase partitioning can be determined by gas chromatography/flame ionization detection (GC-FID). Alkanogenic host cells capable of increased intracellular alkane efflux (due to functional expression of the appropriate heterologous efflux protein apparatus, described herein) should yield higher concentrations of alkane in the solvent layer than an isogenic alkanogenic host cell not expressing said heterologous efflux protein apparatus.

More specifically, to extract a raw culture that is 30 ml in volume (for example), the entire 30 ml contents is poured into a 50 ml tube. 10 ml of iso-octane containing butylated hydroxytolune (BHT) as antioxidant plus an n-eicosane internal standard (IBE) is then added to the emptied flask. After swirling around the 10 ml IBE around the flask to extract any alkane bio-products that may be adherent to the flask interior, the IBE is poured into the aforementioned tube containing the 30 ml raw culture. Then the emptied flask is extracted a second time in the same fashion using a fresh 5 ml volume of IBE to extract any remaining hydrocarbons; this 5 ml is pooled into the tube containing the raw culture and the first 10 ml IBE extract. The entire 30 ml culture/15 ml IBE mixture is then vortexed for 60 seconds, and centrifuged for 15 minutes at 6000 rpm to obtain a clean phase partition between the upper IBE layer and lower aqueous layer, the cells being pelleted at the bottom of the tube. 0.8 ml of the IBE later is then submitted to GC-FID. n-Alkane concentrations in biological IBE extracts are calculated using calibration relationships between GC-FID peak area and known concentrations of authentic n-alkane standards. Knowing the volume of the IBE extractant, the measured concentrations of the n-alkane species in the extractant, and the amount of cells extracted (essentially the product of culture volume and OD₇₃₀), the level of cell-amount-normalized n-alkanes can be determined. Note that the same experimental protocol can be followed using centrifuge-clarified, cell-free spent culture medium instead of raw culture.

Additional embodiments are described in the claims.

INFORMAL SEQUENCE LISTING

SEQ ID NO: 9
sll0041_Nin_PLS_YbhR chimeric protein
MqaptqsgglslrnkAVLIALLIGLIPAGVIGGLNLSsvdrlpvpqteqqvkds
ttkqirdqILIGLLVTAVGAAFVAYWMVGentkaqtalalkakFHRLWILIRKE
LQSLLREPQTRAILILPVLIQVILFPFAATLEVTNATIAIYDEDNGEHSVELTQ
RFARASAFTHVLLLKSPQEIRPTIDTQKALLLVRFPADFSRKLDTFQTAPLQLI
LDGRNSNSAQIAANYLQQIVKNYQQELLEGKPKPNNSELVVRNWYNPNLDYKWF
VVPSLIAMITTIGVMIVTSLSVAREREQGTLDQLLVSPLTTWQIFIGKAVPALI
VATFQATIVLAIGIWAYQIPFAGSLALFYFTMVIYGLSLVGFGLLISSLCSTQQ
QAFIGVFVFMMPAILLSGYVSPVENMPVWLQNLTWINPIRHFTDITKQIYLKDA
SLDIVWNSLWPLLVITATTGSAAYAMFRRKVM
SEQ ID NO: 10
slr1044_Nin_PLS_YbhR chimeric protein
MflgwftnaslfrkqIYMAIASGVFSGFAVLVLGSIVGLGgtpkdvpapsgett
teapaegapaegqapsqtpeeepgkpSLLNLAFLTAIATAIGVFLINrllmqqi
ksiiddlqFHRLWTLIRKELQSLLREPQTRAILILPVLIQVILFPFAATLEVTN
ATIAIYDEDNGEHSVELTQRFARASAFTHVLLLKSPQEIRPTIDTQKALLLVRF
PADFSRKLDTFQTAPLQLILDGRNSNSAQIAANYLQQIVKNYQQELLEGKPKPN
NSELVVRNWYNPNLDYKWFVVPSLIAMITTIGVMIVTSLSVAREREQGTLDQLL
VSPLTTWQIFIGKAVPALIVATFQATIVLAIGIWAYQIPFAGSLALFYFTMVIY
GLSLVGFGLLISSLCSTQQQAFIGVFVFMMPAILLSGYVSPVENMPVWLQNLTW
INPIRHFTDITKQIYLKDASLDIVWNSLWPLLVITATTGSAAYAMFRRKVM
SEQ ID NO: 11
slr1041_Nin_PLS_YbhS chimeric protein
MqaptqsgglslrnkAVLIALLIGLIPAGVIGGLNLSsvdrlpvpqteqqvkds
ttkqirdqILIGLLVTAVGAAFVAYWMVGentkaqtalalkakSNPILSWRRVR
ALCVKETRQIVRDPSSWLIAVVIPLLLLFIFGYGINLDSSKLRVGILLEQRSEA
ALDFTHTMTGSPYIDATISDNRQELIAKMQAGKIRGLVVIPVDFAEQMERANAT
APIQVITDGSEPNTANFVQGYVEGIWQIWQMQRAEDNGQTFEPLIDVQTRYWFN
PAAISQHFIIPGAVTIIMTVIGAILTSLVVAREWERGTMEALLSTEITRTELLL
CKLIPYYFLGMLAMLLCMLVSVFILGVPYRGSLLILFFISSLFLLSTLGMGLLI
STITRNQFNAAQVALNAAFLPSIMLSGFIFQIDSMPAVIRAVTYIIPARYFVST
LQSLFLAGNIPVVLVVNVLFLIASAVMFIGLTWLKTKRRLD
SEQ ID NO: 12
slr1044_Nin_PLS_YbhS chimeric protein
MflgwftnaslfrkqIYMAIASGVFSGFAVLVLGSIVGLGgtpkdvpapsgett
teapaegapaegqapsqtpeeepgkpSLLNLAFLTAIATAIGVFLINrllmqqi
ksiiddlqSNPILSWRRVRALCVKETRQIVRDPSSWLIAVVIPLLLLFIFGYGI
NLDSSKLRVGILLEQRSEAALDFTHTMTGSPYIDATISDNRQELIAKMQAGKIR
GLVVIPVDFAEQMERANATAPIQVITDGSEPNTANFVQGYVEGIWQIWQMQRAE
DNGQTFEPLIDVQTRYWFNPAAISQHFIIPGAVTIIMTVIGAILTSLVVAREWE
RGTMEALLSTEITRTELLLCKLIPYYFLGMLAMLLCMLVSVFILGVPYRGSLLI
LFFISSLFLLSTLGMGLLISTITRNQFNAAQVALNAAFLPSIMLSGFIFQIDSM
PAVIRAVTYIIPARYFVSTLQSLFLAGNIPVVLVVNVLFLIASAVMFIGLTWLK
TKRRLD

What is claimed is:

1. A method for modifying a heterologous integral plasma membrane protein (HIPMP) to improve its functionality in a target cyanobacterial cell, wherein said method comprises:
(i) fusing a pseudo leader sequence (PLS) to the N-terminus of said HIPMP, wherein said HIPMP has, in its native state, its N-terminus within the cytoplasm, and wherein said PLS consists of two transmembrane alpha helices and a single periplasmic loop sequence linking the two transmembrane alpha helices; or
(ii) adding a PLS to the N-terminus of said HIPMP, wherein said HIPMP has, in its native state, its N-terminus within the periplasm, and wherein said PLS consists of a single transmembrane alpha helix.

2. The method of claim 1, wherein said PLS consists of two transmembrane alpha helices and a single periplasmic loop sequence linking the two transmembrane alpha helices, and wherein said PLS is at least 90% identical to a pair of transmembrane alpha helices of an integral plasma membrane protein (IPMP) native to a non-target cyanobacterial species, wherein said IPMP and said pair of transmembrane alpha helices each has, in its native state, its N-terminus within the cytoplasm and its C-terminus within the cytoplasm.

3. The method of claim 1, wherein said PLS consists of a single transmembrane alpha helix that is at least 90% identical to a second transmembrane alpha helix of an IPMP native to a non-target cyanobacterial species, wherein said IPMP and said second transmembrane alpha helix each has, in its native state, its N-terminus within the cytoplasm and its C-terminus within the periplasm.

4. A chimeric integral plasma membrane protein (CIPMP) for facilitating hydrocarbon efflux by a target photosynthetic microorganism, wherein said CIPMP comprises, at its N-terminus, a pseudo leader sequence, wherein said pseudo leader sequence is covalently fused to a heterologous integral plasma membrane protein (IPMP), and wherein said pseudo leader sequence comprises at least one but no more than two transmembrane alpha helices, and wherein the N-terminus of said CIPMP is in the cytoplasm when expressed in said target photosynthetic microorganism.

5. The CIPMP of claim 4, wherein said pseudo leader sequence is identical or homologous to one or two transmembrane alpha helices from a non-target bacterial IPMP.

6. The chimeric protein of claim 5, wherein said IPMP is at least 90% identical to a non-cyanobacterial IPMP and wherein said pseudo leader sequence is at least 90% identical to a non-target cyanobacterial integral IPMP.

7. The chimeric protein of claim 4, wherein the IPMP, in its native state, has its N-terminus in the cytoplasm, and wherein the pseudo leader sequence comprises two transmembrane alpha helices and a periplasmic loop.

8. The chimeric protein of claim 4, wherein the IPMP, it its native state, has its N-terminus in the periplasm, and wherein the pseudo leader sequence comprises a single transmembrane alpha helix.

9. The chimeric protein of claim 8, wherein said IPMP is a non-cyanobacterial integral plasma membrane protein native to Escherichia coli.

10. The chimeric protein of claim 9, wherein said IPMP is a non-cyanobacterial integral plasma membrane protein native to Escherichia coli.

11. The chimeric protein of claim 9 wherein said non-cyanobacterial integral plasma membrane protein is selected from the group consisting of YbhR and YbhS.

12. The chimeric protein of claim 10 wherein said non-cyanobacterial integral plasma membrane protein is selected from the group consisting of YbhR and YbhS.

13. A recombinant nucleic acid encoding the CIPMP of claim 4.

14. A vector comprising a promoter operatively linked to a nucleic acid encoding any of the proteins of claim 4.

15. An engineered photosynthetic microorganism comprising the CIPMP of claim 4.

16. The engineered photosynthetic microorganism of claim 15, further comprising one or more recombinant genes encoding an acyl-ACP reductase enzyme, an alkanal deformylative monooxygenase enzyme, or both enzymes.

17. A method for producing a hydrocarbon, comprising
(i) culturing an engineered photosynthetic microorganism of claim 15 in a culture medium; and
(ii) exposing said engineered photosynthetic microorganism to light and inorganic carbon, wherein said exposure results in the conversion of said carbon dioxide by said engineered photosynthetic microorganism into n-alkanes, wherein said n-alkanes are effluxed into said culture medium in an amount greater than that secreted by an otherwise identical photosynthetic microorganism, cultured under identical conditions, but lacking any of the CIPMP of claim 4.
